SEQUENCE LISTING (14 sequences)

SEQ ID NO: 1 - DNA, 20 bp, Artificial Sequence
  Description of Artificial Sequence Synthetic primer
gtagctgaccatggtgagcg

SEQ ID NO: 2 - DNA, 18 bp, Artificial Sequence
  Description of Artificial Sequence Synthetic primer
aagcagacaccagagcag

SEQ ID NO: 3 - DNA, 18 bp, Artificial Sequence
  Description of Artificial Sequence Synthetic primer
ccctgagccatatcctgt

SEQ ID NO: 4 - DNA, 20 bp, Artificial Sequence
  Description of Artificial Sequence Synthetic primer
cctccgaaaccatgaacttt

SEQ ID NO: 5 - DNA, 20 bp, Artificial Sequence
  Description of Artificial Sequence Synthetic primer
agagatctggttcccgaaac

SEQ ID NO: 6 - DNA, 23 bp, Artificial Sequence
  Description of Artificial Sequence Synthetic primer
caagtggccagaggcatggagtt

SEQ ID NO: 7 - DNA, 24 bp, Artificial Sequence
  Description of Artificial Sequence Synthetic primer
gatgtagtctttaccatcctgttg

SEQ ID NO: 8 - DNA, 22 bp, Artificial Sequence
  Description of Artificial Sequence Synthetic primer
gagggcctctcatggtgattgt

SEQ ID NO: 9 - DNA, 23 bp, Artificial Sequence
  Description of Artificial Sequence Synthetic primer
tgccagcagtccagcatggtctg

SEQ ID NO: 10 - DNA, 20 bp, Artificial Sequence
  Description of Artificial Sequence Synthetic primer
aggagaagctgtgctacgtc

SEQ ID NO: 11 - DNA, 20 bp, Artificial Sequence
  Description of Artificial Sequence Synthetic primer
aggggccggactcgtcatac

SEQ ID NO: 12 - DNA, 20 bp, Artificial Sequence
  Description of Artificial Sequence Synthetic primer
atcagcgtgtgagcacggtg

SEQ ID NO: 13 - DNA, 17 bp, Artificial Sequence
  Description of Artificial Sequence Synthetic primer
gcatctccttttctgac

SEQ ID NO: 14 - DNA, 17 bp, Artificial Sequence
  Description of Artificial Sequence Synthetic primer
ttaatcgttctctgccc

(19) United States
(12) Patent Application Publication
(10) Pub. No.: US 2009/0087424 A1
(43) Pub. Date: Apr. 2, 2009

(54) Modulating Retinal Pigmented Epithelium Permeaion By Inhibiting Or Activating VEGFR-1

(76) Inventors: Noriko Miyamoto, Hyogo (JP); Nadia Normand, Boulogne (FR); Jean Plouet, Paris (FR); Frandine Behar-Cohen, Paris (FR)

Correspondence Address: YOUNG & THOMPSON, 209 Madison Street, Suite 500, ALEXANDRIA, VA, 22314, US

(21) Appl. No.: 11/791,759

(22) PCT Filed: Nov. 26, 2004
(86) PCT No.: PCT/IB04/04107

§ 371 Date: Dec. 11, 2007

(51) Int. Cl. A61K 31/7088; A61K 38/18; A61P 27/02; A61K 39/395

(52) U.S. Cl. 424/130.1; 514/12; 514/44

(57) ABSTRACT

Described is a method for use of an agonist or an antagonist of VEGFR-1 in the treatment or prevention of diseases wherein disorders of the retinal pigment epithelium permeation are involved.

The present invention relates to a method for modulating the permeability of the retinal pigmented epithelial layer. It also relates to a method for treating or preventing various diseases associated with permeability disorders of the retinal pigmented epithelial layer (RPE).

### BACKGROUND OF INVENTION

VEGF-A (vascular endothelial growth factor) is an homodimeric protein that contains one of the five VEGF isoforms: 121, 145, 165, 189 or 206 amino acids (VEGF-121, -145, -165, -189, or -206) (Ferrara et al. Nat. Med. 2003 June; 9(6):669-76. Review; Gerber et al., J Biol Chem. 1997 Sep. 19; 272(38):23659-67). VEGF receptors have been identified in vascular endothelial cells and in pericytes (Witmer et al., Prog Retin Eye Res. 2003 January; 22(1):1-29. Review). Two high affinity receptors for VEGF, named VEGFR-1 or Flt-1 and VEGFR-2 or KDR (Flk-1 in mouse) have been characterised. They are both transmembrane proteins with cytoplasmic tyrosine kinase domains, and are expressed on endothelial cells. The two receptors appear to mediate different biological effects of VEGF. Mice lacking the complete Flt-1 gene display an increased number of endothelial progenitors and vascular disorganization and die in utero at embryonic day 9 (Fong et al. Nature 1995, 376:66-70). Mice in which the flk-1/KDR gene has been inactivated also die at embryonic day 9. They are deficient in vasculogenesis and also lack blood island formation (Shalaby et al. Nature 1995, 376:62-66).

Placental growth factor (PlGF) is an homodimeric protein that shares substantial structural similarity with VEGF-A. Three PlGF isoforms have been described (PlGF-1, -2 and-3). These isoforms do not interact with VEGFR-2 but do bind to VEGFR-1 (Christinger et al, J Biol. Chem. 2004 Mar. 12; 279(11):10382-8. Epub 2003 Dec. 18.).

It is generally accepted that activation of VEGFR-2 induces angiogenesis and endothelial cell proliferation (Millauer et al., Nature. 1994 Feb. 10; 367(6463):576-9; Jonca et al., J Biol. Chem. 1997 Sep. 26; 272(39):24203-9) while activation of VEGFR-1 mostly induces migration of endothelial cells (Barleon et al., Blood. 1996 Apr. 15; 87(8):3336-43) and monocytes (Barleon et al., 1996; Autiero et al., J Thromb Haemost. 2003 July; 1(7):1356-70. Review).

It has been demonstrated that VEGF plays an important role in neovascular diseases in animal models and in humans. VEGF enhances pathological angiogenesis in synergy with other cytokines, and induces vascular permeability (Witmer et al., 2003; Caldwell et al, Diabetes Metab Res Rev. 2003 November-December; 19(6):442-55. Review). In vivo, VEGF potently increases microvascular permeability of post capillary venules and capillaries, and can rapidly induce fenestrations in continuous endothelia when injected in skin or muscle. This effect on endothelial permeability is probably involved in the pathogenesis of malignant ascites, ovarian hyperstimulation syndrome and oedema around brain tumours, bullous skin diseases, and psoriasis.

The role of VEGFR-1 in angiogenesis was studied by several groups. Marchand et al. demonstrated that antisenses sequence directed against Flt-1 (SEQ ID No2: 5′-AAGCAGACACCAGAGCAG-3′ and SEQ ID No3: 5′-CCCTGAGCCATATCCTGT-3′) reduced by ˜80% the VEGF-induced formation of new blood vessels in mouse testis (Marchand et al., Am J Physiol Heart Circ Physiol. 2002 January; 282(1):H194-204).

In vitro, a chimeric protein containing the soluble form of VEGFR-1 receptor (sFlt-1) suppressed the angiogenic growth capacity of endothelial cells from patients with rheumatoid arthritis (Sekimoto et al., J. Rheumatol. 2002 February; 29(2):240-5). Treatment with sFlt-1, either transfected or administered by itself, proved its therapeutic efficacy antagonizing VEGF in various models of ovarian tumors (Mahasreshti et al., Clin Cancer Res. 2001 July; 7(7):2057-66, Hasumi et al., Cancer Res. 2002 Apr. 1; 62(7):2019-23), thyroid carcinoma (Ye et al., Endocrinology. 2004 February; 145(2):817-22), lung metastasis (Yoshimura et al., J Urol. 2004 June; 171(6 Pt 1):2467-70). Also monoclonal antibodies directed against VEGFR-1 inhibited the growth of C6 glioma in a mouse xenograft (Stefanik et al., J. Neurooncol. 2001 November; 55(2):91-100). Gene transfer of VEGFR-1 mutants successfully decreased vascular density and tumor cell proliferation in vivo (Heidenreich et al., Int J. Cancer. 2004 Sep. 1; 111(3):348-57). In addition, An et al. (Int J. Cancer. 2004 Aug. 20; 111(2):165-73) recently demonstrated in vivo successful tumor growth and metastasis inhibition by administration of an enzyme-bound VEGFR-1 antagonizing peptide.

The potential of this approach is also reflected by the numerous applied patents.

For example, U.S. Pat. No. 6,383,486 describes novel chimeric VEGF receptor proteins comprising amino acid sequences derived from the vascular endothelial growth factor (VEGF) receptor flt-1 which antagonize the endothelial cell proliferative and angiogenic activity allowing utilizing such proteins for the treatment of conditions associated with undesired vascularization.

U.S. Pat. No. 6,057,428 describes the modification of the kinase domain region of VEGFR-1 such that the binding affinity to native VEGF is modified.

U.S. Pat. No. 6,375,929 describes the use of gene therapy for expressing soluble VEGFR-1 form for inhibiting angiogenesis associated with solid tumor growth, tumor metastasis, inflammation, psoriasis, rheumatoid arthritis, hemangiomas, diabetic retinopathy, angiofibromas, and macular degeneration.

U.S. Pat. No. 6,245,512 describes a method for regulating VEGFR-1 concentration by regulating the activity of the receptor promoter.

U.S. Pat. Nos. 6,365,157 and 6,448,077 describe monoclonal, chimeric and humanized antibodies that specifically bind to the VEGFR-1 receptor and neutralize activation of the receptor, providing a method for reducing tumor growth in a mammal.

U.S. Pat. No. 6,114,320 discloses a method using a selective PKC inhibitor for inhibiting VEGF stimulated endothelial cell growth, such as associated with macular degeneration, and VEGF stimulated capillary permeability, such as associated with macular edema.

U.S. Pat. No. 6,617,160 describes a monoclonal antibody which immunologically reacts with human VEGF receptor Flt-1 and inhibits its binding, providing means for the diagnosis or treatment of diseases such as proliferation or metastasis of solid tumors, arthritis in rheumatoid, arthritis, diabetic retinopathy, retinopathy of prematurity and psoriasis.

Disclosed and claimed in U.S. Pat. No. 6,696,252 are antisense sequences capable of modulate indistinctinly VEGFR-1 and VEGFR-2. Disclosed and claimed in U.S. Pat. No. 6,710,174 are antisense sequences capable of regulating VEGFR-1.

Despite all the above mentioned studies, there is still significant uncertainty concerning the role of individual VEGF receptors for other biological effects than mitogenesis, such as permeability.

Stacker et al. show that a VEGF mutant chimeric protein that is unable to trigger VEGFR-2 activation can cause vascular permeability to an extent that is similar to wild type VEGF, concluding that Flt-1 could be responsible for the increased VEGF-induced vascular permeability (Stacker et al., J Biol. Chem. 1999 Dec. 3; 274(49):34884-92). Gille et al. reported that while a selective VEGFR-2 agonist induced comparable vascular permeability as the wild type VEGF, the VEGFR-1 selective VEGF caused essentially no leakage even at high concentrations in the rat cornea. Also in dye extravasation tests a known VEGFR-1 agonist (PlGF) failed to induce any vascular permeability (Gille et al., J Biol. Chem. 2001 Feb. 2; 276(5):3222-30).

A recent report describes the increased vascular permeability induced by a protein originated from snake venom which has VEGFR-1 agonist activity but binds to VEGFR-2 weakly (Takahashi et al., J Biol. Chem. 2004 Oct. 29; 279 (44):46304-14).

Angiogenesis plays also a crucial role in disorders responsible for most blind registration in the Western world. VEGF has been shown particularly to be associated with numerous ocular pathologic conditions associated with angiogenesis, including the pathogenesis of diabetic retinopathy (DR), retinopathy of prematurity (ROP), age-related macular degeneration (ARMD) and ocular neovascularization (ON). VEGF is produced by vascular retinal endothelial cells and pericytes, retinal pigment epithelial cells (RPE), astocytes and retinal Müller glial cells (RMG) (Simorre-Pinatel et al, Invest Opthalmol V is Sci. 1994 August; 35(9):3393-400; Adamis et al., Biochem Biophys Res Commun. 1993 Jun. 15; 193(2):631-8; Behzadian et al., Glia 1998 October; 24(2):216-25).

VEGF receptors have been identified in RPE cells, inner retina and in synaptic complexes of cone-photoreceptors (Witmer et al., 2003). VEGFR-1 has been identified as the predominant VEGF receptor in pericyte microvessels of normal human and monkey adult retinas (Witmer et al., 2003; Witmer et al., Invest Opthalmol Vis Sci. 2002 March; 43(3):849-57). It is also present in the inner retina (Witmer et al., 2003) and in human RPE cells (Ohno-Matsui et al., Biochem Biophys Res Commun. 2003 Apr. 11; 303(3):962-7).

In ophthalmology, inhibition of angiogenesis was also proved to be of great value. Injection or transfection with sFlt-1 in the anterior chamber inhibited corneal and choroidal angiogenesis in animal models of neovascularization (Lai et al., Hum Gene Ther. 2001 Jul. 1; 12(10):1299-310, Exp Eye Res. 2002 December; 75(6):625-34). Antibodies to the receptor demonstrated as well therapeutic potential suppressing neovascularization in ischemic retina (Luttun et al., Nat. Med. 2002 August; 8(8):831-40) and Rota et al. showed in the rat model of ROP impressive reduction in retinal neovascularization following intravitreal injection of an adenoviral vector expressing sFlt-1 (Rota et al., J Gene Med. 2004 September; 6(9):992-1002).

The role of VEGFR-1 on the retinal pigmented epithelial barrier has however never been elucidated. The inventors have discovered the role of VEGFR-1 on the retinal permeability.

Consequently the instant invention concerns the potential applications of agonists or antagonists of VEGFR-1 in the ophthalmic domain for regulating the external hemato-retinal barrier.

### DESCRIPTION OF THE INVENTION

The present invention relates to the use of an agonist or an antagonist of vascular endothelial growth factor receptor 1 (VEGFR-1) for the manufacture of a medicament for preventing or treating a subject suffering from an ocular disease wherein disorders of the retinal pigment epithelium (RPE) permeation are involved. According to the instant invention, the ocular disease wherein disorders of the RPE permeation are involved is selected from the group comprising macular edema of any origin such as those associated or not to serous detachment of RPE cells, central serous chorioretinopathy, epitheliopathy of any origin, age related macular degeneration (ARMD) such as ARMD with leakage from the choriocapillaries into the subretinal space.

The expression “agonist of VEGFR-1” refers in the present invention to any compound which, when it binds to VEGFR-1, increases RPE cell permeability by at least 25%, preferably 30, 40, 50, 60, 70 or 80%.

Cellular permeability can be assessed by transepithelial electrical resistance (TER) and tritiated inulin flux across RPE monolayers (see point 1.1 of the examples).

In a specific embodiment of the invention, the agonist of VEGFR-1 is selected from the group comprising vascular endothelial growth factor (VEGF), placenta growth factor (PlGF) including PlGF1, agonists originating from snake venoms such as from Trimeresurus flavoridis venom, and specific agonists found by the idiotypic technologies.

In a more advantageous embodiment of the invention, the agonist is the placenta growth factor (PlGF), preferably PlGF1.

The expression “antagonist of VRGFR-1” refers in the present invention to any compound which, when it binds to VEGFR-1, reduces RPE cell permeability by at least 25%, preferably 30, 40, 50, 60, 70 or 80%. As described above, cellular permeability can be assessed by TER and tritiated inulin flux across RPE monolayers.

In another specific embodiment of the invention, the antagonist of VEGFR-1 is selected from the group comprising blocking antibodies such as MF1 antifltlmab, peptides such as DHFR-F56/F90 (Lei. H et al. Zhonghua Yi Xue Za Zhi. 2002 Oct. 10; 82(19):1342-5), oligoaptamer, ribozymes, siRNA, antisense oligonucleotides, or compounds such as immunomodulator compounds, for example FTY720 (Sanchez et al., J Biol. Chem. 2003 Nov. 21; 278(47):47281-90), 5-[N-methyl-N-(4-octadecyloxyphenyl)-acetyl]amino-2-methylthiobenzoic acid (Ueda et al., Mol Cancer Ther. 2003 November; 2(11):1105-11) and acetyl salicilate.

Preferably, the antagonist is an antisense oligonucleotide of the mRNA encoding VEGFR-1, said antisense oligonucleotide having the following sequence:
a) the sequence SEQ ID No1, or
b) a sequence having at least 70, 72, 75, 77, 80, 82, 85, 87, 90, 91, 92, 93, 94, 95, 96, 97, 98 or 99% of identity with SEQ ID No1, or
c) a sequence comprising the sequence as defined in a) or b), or
d) a sequence complementary to the sequence as defined in a), b) or c).

In the context of this invention, the term “oligonucleotide” refers to an oligomer or polymer of ribonucleic acid (RNA) or deoxyribonucleic acid (DNA) or mimetics thereof. This term includes oligonucleotides composed of naturally-occurring nucleobases, sugars and covalent internucleoside (backbone) linkages as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases.

While antisense oligonucleotides are a preferred form of antisense compound, the present invention comprehends other oligomeric antisense compounds, including but not limited to oligonucleotide mimetics such as are described below. The antisense compounds in accordance with this invention preferably comprise from about 8 to about 50 nucleobases (i.e. from about 8 to about 50 linked nucleosides). Particularly preferred antisense compounds are antisense oligonucleotides, even more preferably those comprising from about 12 to about 30 nucleobases. Antisense compounds include ribozymes, external guide sequence (EGS) oligonucleotides (oligozymes), and other short catalytic RNAs or catalytic oligonucleotides which hybridize to the target nucleic acid and modulate its expression.

As known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to the 2′, 3′ or 5′ hydroxyl moiety of the sugar. In forming oligonucleotides, the phosphate groups covalently link adjacent nucleosides to one another to form a linear polymeric compound. In turn the respective ends of this linear polymeric structure can be further joined to form a circular structure, however, open linear structures are generally preferred. Within the oligonucleotide structure, the phosphate groups are commonly referred to as forming the internucleoside backbone of the oligonucleotide. The normal linkage or backbone of RNA and DNA is a 3′ to 5′ phosphodiester linkage.

Specific examples of preferred antisense compounds useful in this invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

Preferred modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and borano-phosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Preferred oligonucleotides having inverted polarity comprise a single 3′ to 3′ linkage at the 3′-most internucleotide linkage i.e. a single inverted nucleoside residue which may be abasic (the nucleobase is missing or has a hydroxyl group in place thereof). Various salts, mixed salts and free acid forms are also included.

Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, certain of which are commonly owned with this application, and each of which is herein incorporated by reference.

Preferred modified oligonucleotide backbones that do not include a phosphorus atom therein have backbones that are formed by short chain allyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH.sub.2 component parts.

Representative United States patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, certain of which are commonly owned with this application, and each of which is herein incorporated by reference. See U.S. Pat. No. 6,710,174 for further information concerning replacement of sugar, internucleoside linkage or nucleobase with respectively novel groups or nucleobases, or concerning oligonucleotide conjugates.

The antisense compounds used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

The antisense compounds of the invention are synthesized in vitro and may include antisense compositions of biological origin, or genetic vector constructs designed to direct the in vivo synthesis of antisense molecules (see for example Current Protocols in Molecular Biology, Volumes I, II, and m, 1997 (F. M. Ausubel ed.); Sambrook et al., 1989, Molecular Cloning: A Laboratory Manual, Second Edition, Cold Spring Harbor Laboratory Press, Cold Spring Harbor, N.Y.; DNA Cloning: A Practical Approach, Volumes I and II, 1985 (D. N. Glover ed.); Oligonucleotide Synthesis, 1984 (M. L. Gait ed.); Nucleic Acid Hybridization, 1985, (Hames and Higgins); Transcription and Translation, 1984 (Hames and Higgins eds.); Animal Cell Culture, 1986 (R. I. Freshney ed.); Immobilized Cells and Enzymes, 1986 (IRL Press); Perbal, 1984, A Practical Guide to Molecular Cloning; the series, Methods in Enzymology (Academic Press, Inc.); Gene Transfer Vectors for Mammalian Cells, 1987 (J. H. Miller and M. P. Calos eds., Cold Spring Harbor Laboratory); and Methods in Enzymology Vol. 154 and Vol. 155 (Wu and Grossman, and Wu, eds., respectively) for conventional techniques in molecular biology, microbiology, and recombinant DNA). The compounds of the invention may also be admixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption.

Representative United States patents that teach the preparation of such uptake, distribution and/or absorption assisting formulations include, but are not limited to, U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

The antisense compounds of the invention encompass any pharmaceutically acceptable salts, esters, or salts of such esters or any other compound which, upon administration to an animal including a human, is capable of providing (directly or indirectly) the biologically active metabolite or residue thereof. Accordingly, for example, the disclosure is also drawn to prodrugs and pharmaceutically acceptable salts of the compounds of the invention, pharmaceutically acceptable salts of such prodrugs, and other bioequivalents.

The term “prodrug” indicates a therapeutic agent that is prepared in an inactive form that is converted to an active form (i.e., drug) within the body or cells thereof by the action of endogenous enzymes or other chemicals and/or conditions. In particular, prodrug versions of the oligonucleotides of the invention are prepared as SATE [(S-acetyl-2-thioethyl) phosphate] derivatives according to the methods disclosed in WO 93/24510 to Gosselin et al., published Dec. 9, 1993 or in WO 94/26764 and U.S. Pat. No. 5,770,713 to Imbach et al.

The term “pharmaceutically acceptable salts” refers to physiologically and pharmaceutically acceptable salts of the compounds of the invention: i.e., salts that retain the desired biological activity of the parent compound and do not impart undesired toxicological effects thereto, and are well known by the man skilled in the art.

By percentage of identity between two nucleic acid or amino acid sequences in the present invention, it is meant a percentage of identical nucleotides or amino acid residues between the two sequences to compare, obtained after the best alignment; this percentage is purely statistical, and the differences between the two sequences are randomly distributed and all along their length. The best alignment or optimal alignment is the alignment corresponding to the highest percentage of identity between the two sequences to compare, which is calculated such as herein after. The sequence comparisons between two nucleic acid or amino acid sequences are usually performed by comparing these sequences after their optimal alignment, said comparison being performed for one segment or for one “comparison window”, to identify and compare local regions of sequence similarity. The optimal alignment of sequences for the comparison can be performed manually or by means of the algorithm of local homology of Smith and Waterman (1981) (Ad. App. Math. 2:482), by means of the algorithm of local homology of Neddleman and Wunsch (1970) (J. Mol. Biol. 48:443), by means of the similarity research method of Pearson and Lipman (1988) (Proc. Natl. Acad. Sci. USA 85:2444), by means of computer softwares using these algorithms (GAP, BESTFIT, FASTA and TFASTA in the Wisconsin Genetics Software Package, Genetics Computer Group, 575 Science Dr., Madison, Wis.).

The percentage of identity between two nucleic acid or amino acid sequences is determined by comparing these two aligned sequences in an optimal manner with a “comparison window” in which the region of the nucleic acid or amino acid sequence to compare may comprise additions or deletions with regard the sequence of reference for an optimal alignment between these two sequences. The percentage of identity is calculated by determining the number of positions for which the nucleotide or the amino acid residue is identical between the two sequences, by dividing this number of identical positions by the total number of positions in the “comparison window” and by multiplying the result obtained by 100, to obtain the percentage of identity between these two sequences.

According to the instant invention, the subject, also named herein “patient”, is a mammal, preferably a human being.

The instant invention also provides a pharmaceutical composition comprising an antisense oligonucleotide of the mRNA encoding VEGFR-1, said antisense oligonucleotide having the following sequence:
a) the sequence SEQ ID No1, or
b) a sequence having at least 70% of identity with SEQ ID No1, or
c) a sequence comprising the sequence as defined in a) or b), or
d) a sequence complementary to the sequence as defined in a), b) or c), together with a pharmaceutically acceptable carrier.

In an embodiment of the invention, the pharmaceutical composition is used for preventing or treating a subject suffering from an ocular disease wherein disorders of the RPE permeation are involved.

In a specific embodiment of the invention, the ocular disease wherein disorders of the RPE permeation are involved is one listed above.

The invention also relates to a method for treating or preventing a subject suffering from an ocular disease wherein disorders of the RPE permeation are involved, comprising administering to said subject a pharmaceutical composition comprising an agonist or an antagonist of VEGFR-1 together with a pharmaceutically acceptable carrier.

The invention further relates to a pharmaceutical composition containing a first compound which is an agonist or an antagonist of VEGFR-1 and a second active compound as a combined preparation for simultaneous, separate or sequential use to treat an ocular disease.

In this case, said agonist or antagonist of VEGFR-1 modulates the intraocular penetration of the second active compound systemically administered.

The second active compound may be an agonist or an antagonist of VEGFR-1 or any compound able to treat an ocular disease, such as for example antibodies, therapeutic proteins, peptides, high molecular weight compounds, viruses, polymeric or peptidic nano- or micro-particles.

In a specific embodiment, the ocular disease is selected from the group comprising macular edema of any origin such a those associated or not to serous detachment of RPE cells, central serous chorioretinopathy, epitheliopathy of any origin, age related macular degeneration (ARMD) such as ARMD with leakage from the choriocapillaries into the subretinal space, infections, intraocular inflammations, retinal dystrophy of any origin, diabetic retinopathy, ischemic retinopathy, optic neuritis, and any ocular diseases associated to neo vascular and/or fibroglial proliferations.

In a preferred embodiment of the invention, the ocular disease is an ocular disease wherein disorders of the RPE permeation are involved.

In a more specific embodiment of the invention, the agonist is selected from the group comprising vascular endothelial growth factor (VEGF), placenta growth factor (PlGF) including PlGF1, agonists originating from snake venoms such as from Trimeresurus flavoridis venom, and specific agonists found by the idiotypic technologies known from the one skilled in the art; advantageously the agonist is the PlGF.

In another preferred embodiment of the invention, the antagonist is selected from the group comprising blocking antibodies such as MF1 antiflt1mAb, peptides such as DHFR-F56/F90 oligoaptamer, ribozymes, siRNA, antisense oligonucleotides, or compounds such as immunomodulator compounds, for example FTY720, 5-[N-methyl-N-(4-octadecyloxyphenyl)-acetyl]amino-2-methylthiobenzoic acid and acetyl salicilate.

The expressions “active compound” or “second active compound” refer in the present invention to any compound which improves the pathological condition of a subject suffering from an ocular disease. Said improvement of pathological condition corresponds to an improvement of at least 25%, preferably 30, 40, 50, 60, 70 or 80% of anyone of signs and symptoms detected when the ocular disease occurs.

According to the instant invention, the second active compound is selected in the group comprising anti angiogenic compounds, non steroidal anti inflammatory, cortico and mineralo corticoids, anti-oxidants, lipids, neurotrophic factors (ciliary neurotrophic factor or CNTF, glial cell line-derived neurotrophic factor or GDNF, brain-derived neurotrophic factor or BDNF, fibroblast growth factor or FGF), COX inhibitors and antibiotics.

Still furthermore the invention relates to the use of a first compound which is an agonist or an antagonist of VEGFR-1 and a second active compound for the preparation of a combined preparation to be administered simultaneously, separately or sequentially for preventing or treating a subject suffering from an ocular disease, the first compound modulating the intraocular penetration of the second active compound systemically administered.

In a specific embodiment of the invention, the ocular disease is selected from the group comprising macular edema of any origin such as those associated or not to serous detachment of RPE cells, central serous chorioretinopathy, epitheliopathy of any origin, age related macular degeneration (ARMD) such as ARMD with leakage from the choriocapillaries into the subretinal space, infections, intraocular inflammations, retinal dystrophy of any origin, diabetic retinopathy, ischemic retinopathy, optic neuritis, and any ocular diseases associated to neo vascular and/or fibroglial proliferations.

In a preferred embodiment, the agonist is selected from the group comprising of VEGF agonists originating from Trimeresurus flavoridis venom, and specific agonists found by the idiotypic technologies.

In another preferred embodiment, the agonist is the PlGF.

In a further specific embodiment of the invention, the second active compound is selected from the group comprising anti angiogenic compounds, non steroidal anti inflammatory, cortico and mineralo corticoids, anti-oxidants, lipids, neurotrophic factors (ciliary neurotrophic factor or CNTF, glial cell line-derived neurotrophic factor or GDNF, brain-derived neurotrophic factor or BDNF, fibroblast growth factor or FGF), COX inhibitors and antibiotics.

In another specific embodiment, the antagonist is selected from the group comprising blocking antibodies such as MF1 anti fltlmAb, peptides such as DHFR-F56/F90, oligoaptamer, ribozymes, siRNA, antisense oligonucleotides, or compounds such as immunomodulator compounds, for example FTY720, 5-[N-methyl-N-(4-octadecyloxyphenyl)-acetyl]amino-2-methylthiobenzoic acid and acetyl salicilate.

The invention also relates to a method for treating or preventing a subject suffering from an ocular disease comprising administering simultaneously, separately or sequentially to said subject a combined pharmaceutical preparation comprising a first compound, which is an agonist or an antagonist of VEGFR-1, and a second active compound together with a pharmaceutically acceptable carrier.

The ocular disease may be of any type as listed above.

The pharmaceutical composition of the invention comprises molecules as described above and a pharmaceutically acceptable carrier or excipient (both terms can be used interchangeably) to treat diseases as indicated above. Suitable carriers or excipients known to the skilled man are saline, Ringer's solution, dextrose solution, Hank's solution, fixed oils, ethyl oleate, 5% dextrose in saline, substances that enhance isotonicity and chemical stability, buffers and preservatives. Other suitable carriers include any carrier that does not itself induce the production of antibodies harmful to the individual receiving the composition such as micelles, emulsions, proteins, polysaccharides, polylactic acids, polyglycolic acids, polymeric amino acids and amino acid copolymers. Further details on techniques for formulation and administration may be found in the latest edition of Remington's Pharmaceutical Sciences (Maack Publishing Co., Easton, Pa.).

The medicament may be administered by any suitable method within the knowledge of the skilled man. One route of administration is parenterally. In parenteral administration, the medicament of this invention will be formulated in a unit dosage injectable form such as a solution, suspension or emulsion, in association with the pharmaceutically acceptable excipients as defined above. The preferred route of administration is ocularly, for example by transcleral delivery of bioreactive protein to the choroid and retina (Ambati et al. (2000) Investigative Opthalmology and Visual Science, 41, 1186). The formulation of topical ophthalmic preparations, including ophthalmic solutions, suspensions and ointments is well known to those skilled in the art (see Remington's Pharmaceutical Sciences, 18th Edition, Chapter 86, pages 1581-1592 Mack Publishing Company, 1990). Other modes of administration are available, including intracameral injections (which may be made directly into the anterior chamber or directly into the vitreous chamber), subconjunctival injections and retrobulbar injections, and methods and means for producing ophthalmic preparations suitable for such modes of administration are also well known.

The term “extraocular” refers to the ocular surface and the (external) space between the eyeball and the eyelid. Examples of extraocular regions include the eyelid fornix or cul-de-sac, the conjunctival surface and the corneal surface. This location is external to all ocular tissues and an invasive procedure is not required to access this region. Examples of extraocular systems include inserts and “topically” applied drops, gels or ointments which may be used to deliver therapeutic material to these regions. Extraocular devices are generally easily removable, even by the patient.

The following patents disclose extraocular systems which are used to administer drugs to the extraocular regions. Higuchi et al. discloses in U.S. Pat. Nos. 3,981,303, 3,986,510 and 3,995,635, a biodegradable ocular insert which contains a drug. The insert can be made in different shapes for retention in the cul-de-sac of the eyeball, the extraocular space between the eyeball and the eyelid. Several common biocompatible polymers are disclosed as suitable for use in fabricating this device. These polymers include zinc alginate, poly (lactic acid), poly (vinyl alcohol), poly (anhydrides) and poly (glycolic acid). The patents also describe membrane coated devices with reduced permeation to the drug and hollow chambers holding the drug formulation.

Theeuwes, U.S. Pat. No. 4,217,898, discloses microporous reservoirs which are used for controlled drug delivery. These devices are placed extraocularly in the ocular cul-de-sac. Among the polymer systems of interest include poly (vinylchloride)-co-poly (vinyl acetate) copolymers. Kaufman discloses in U.S. Pat. Nos. 4,865,846 and 4,882,150 an ophthalmic drug delivery system which contains at least one bio-erodible material or ointment carrier for the conjunctival sac. The patent discloses polymer systems, such as, poly (lactide), poly (glycolide), poly (vinyl alcohol) and cross linked collagen, as suitable delivery systems.

It is also advantageous that a topically applied ophthalmic formulation includes an agent to promote the penetration or transport of the therapeutic agent into the eye. Such agents are known in the art. For example, Ke et al., U.S. Pat. No. 5,221,696 discloses the use of materials to enhance the penetration of ophthalmic preparations through the cornea.

Intraocular systems are those systems which are suitable for use in any tissue compartment within, between or around the tissue layers of the eye itself. These locations include subconjunctival (under the ocular mucous membrane adjacent to the eyeball), orbital (behind the eyeball), and intracameral (within the chambers of the eyeball itself). In contrast to extraocular systems, an invasive procedure consisting of injection or implantation is required to access these regions.

The following patents disclose intraocular devices. Wong, U.S. Pat. No. 4,853,224, discloses microencapsulated drugs for introduction into the chamber of the eye. Polymers which are used in this system include polyesters and polyethers. Lee, U.S. Pat. No. 4,863,457, discloses a biodegradable device which is surgically implanted intraocularly for the sustained release of therapeutic agents. The device is designed for surgical implantation under the conjunctiva (mucous membrane of the eyeball). Krezancaki, U.S. Pat. No. 4,188,373, discloses a pharmaceutical vehicle which gels at human body temperature. This vehicle is an aqueous suspension of the drug and gums or cellulose derived synthetic derivatives. Haslam et al. discloses in U.S. Pat. Nos. 4,474,751 and 4,474,752 a polymer-drug system which is liquid at room temperature and gels at body temperature. Suitable polymers used in this system include polyoxyethylene and polyoxy propylene. Davis et al. disclose in U.S. Pat. No. 5,384,333 a biodegradable injectable drug delivery polymer which provides long term drug release. The drug composition is made up of a pharmaceutically active agent in a biodegradable polymer matrix, where the polymer matrix is a solid at temperatures in the range 20.degree. to 37.degree. C. and is flowable at temperatures in the range 38.degree. to 52.degree. C. The drug delivery polymer is not limited to the delivery of soluble or liquid drug formulations. For example, the polymer can be used as a matrix for stabilizing and retaining at the site of injection drug-containing microspheres, liposomes or other particulate-bound drugs.

A particularly suitable vehicle for intraocular injection is sterile distilled water in which one of the agonist of VEGFR-1, antagonist of VEGFR-1 and combined preparation VEGFR-1 agonist/second active compound is formulated as a sterile, isotonic solution, properly preserved. Yet another ophthalmic preparation may involve the formulation of such agonist, antagonist or combined preparation with an agent, such as injectable microspheres or liposomes, which provides for slow or sustained release. Other suitable means for the intraocular introduction include implantable drug delivery devices.

The ophthalmic preparations of the present invention, particularly topical preparations, may include other components, for example ophthalmically acceptable preservatives, tonicity agents, cosolvents, wetting agents, complexing agents, buffering agents, antimicrobials, antioxidants and surfactants, as are well known in the art. For example, suitable tonicity enhancing agents include alkali metal halides (preferably sodium or potassium chloride), mannitol, sorbitol and the like. Sufficient tonicity enhancing agent is advantageously added so that the formulation to be instilled into the eye is hypotonic or substantially isotonic. Suitable preservatives include, but are not limited to, benzalkonium chloride, thimerosal, phenethyl alcohol, methylparaben, propylparaben, chlorhexidine, sorbic acid and the like. Hydrogen peroxide may also be used as preservative. Suitable cosolvents include, but are not limited to, glycerin, propylene glycol and polyethylene glycol. Suitable complexing agents include caffeine, polyvinylpyrrolidone, beta-cyclodextrin or hydroxypropyl-beta-cyclodextrin. Suitable surfactants or wetting agents include, but are not limited to, sorbitan esters, polysorbates such as polysorbate 80, tromethamine, lecithin, cholesterol, tyloxapol and the like. The buffers can be conventional buffers such as borate, citrate, phosphate, bicarbonate, or Tris-HCl.

The formulation components are present in concentrations that are acceptable to the extraocular or intraocular site of administration. For example, buffers are used to maintain the composition at physiological pH or at slightly lower pH, typically within a pH range of from about 5 to about 8. Additional formulation components may include materials which provide for the prolonged ocular residence of the extraocularly administered therapeutic agent so as to maximize the topical contact and promote absorption. Suitable materials include polymers or gel forming materials which provide for increased viscosity of the ophthalmic preparation. Chitosan is a particularly suitable material as an ocular release-rate controlling agent in sustained release liquid ophthalmic drug formulations (see U.S. Pat. No. 5,422,116, Yen, et. al.) The suitability of the formulations of the instant invention for controlled release (e.g., sustained and prolonged delivery) of an ophthalmic treating agent in the eye can be determined by various procedures known in the art, e.g., as described in Journal of Controlled Release, 6:367-373, 1987, as well as variations thereof.

Pharmaceutical compositions suitable for use in the invention include compositions wherein the active ingredients are contained in an effective amount to achieve the intended purpose. The determination of an effective dose is well within the capability of those skilled in the art.

For any compound, the therapeutically effective dose can be estimated initially either in cell culture assays, e.g., of neoplastic cells, or in animal models, usually mice, rabbits, dogs, or pigs. The animal model may also be used to determine the appropriate concentration range and route of administration. Such information can then be used to determine useful doses and routes for administration in humans.

Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio between toxic and therapeutic effects is the therapeutic index, and it can be expressed as the ratio, LD50/ED50. Pharmaceutical compositions which exhibit large therapeutic indices are preferred. The data obtained from cell culture assays and animal studies is used in formulating a range of dosage for human use. The dosage contained in such compositions is preferably within a range of circulating concentrations that include the ED50 with little or no toxicity. The dosage varies within this range depending upon the dosage form employed, sensitivity of the patient, and the route of administration.

The exact dosage will be determined by the practitioner, in light of factors related to the subject that requires treatment. Dosage and administration are adjusted to provide sufficient levels of the active moiety or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

Preferably, the medicament is administered so that the molecule of the present invention is given at a dose between 1 μg/kg and 10 mg/kg, more preferably between 10 μg/kg and 5 mg/kg, most preferably between 0.1 and 2 mg/kg. Preferably, it is given as a bolus dose. Continuous infusion may also be used and includes continuous subcutaneous delivery via an osmotic minipump. If so, the medicament may be infused at a dose between 5 and 20 μg/kg/minute, more preferably between 7 and 15 μg/kg/minute. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc. Pharmaceutical formulations suitable for oral administration of proteins are described, e.g., in U.S. Pat. Nos. 5,008,114; 5,505,962; 5,641,515; 5,681,811; 5,700,486; 5,766,633; 5,792,451; 5,853,748; 5,972,387; 5,976,569; and 6,051,561.

The invention will be fully illustrated by the following figures and examples. All of the starting materials and reagents disclosed below are known to those skilled in the art, and are available commercially or can be prepared using well known techniques.

### BRIEF DESCRIPTION OF THE FIGURES

FIG. 1: Effect of VEGF on transepithelial electrical resistance (TER) and transepithelial flux of tritiated inulin of cultured ARPE-19 as a function of time. Confluent ARPE-19 cells grown on transwell polyester filters for 4 weeks form monolayers with constant TER. These monolayers were incubated with various concentrations of VEGF. Paracellular permeability was assessed by TER recording or apical to basal [³H] inulin flux measurements.

FIG. 1A: Time course of TER decrease after incubation with 0.2, 0.5 and 1 nM VEGF. Net TER results from the difference between 10 measures of TER cell monolayer and TER filter alone. Results are expressed as percentage of net TER in comparison with net TER before exposure to VEGF. Mean±SD % TER result from three separate experiments.

FIG. 1B: Effect of mM on TER ARPE-19 monolayers measured at 6, 24, 36, 48 and 65 hours.

FIG. 1C: Time course of inulin flux across ARPE-19 monolayers after incubation with 1 nM VEGF. ³H-inulin flux was measured by counting radioactivity (indicated by cpm) in 10 μl during a period of 10 to 180 minutes. Data are representative of 3 experiments.

FIG. 1D: Inulin flux across RPE monolayer 10 minutes after incubation of various concentration of VEGF ranging from 0.1 to 1 nM VEGF.

FIG. 2: Effect of 1 nM VEGF on occludin distribution as shown by immunohistochemistry on ARPE-19 monolayers at 20 min, 24 hours and 65 hours. Fluorescence microphotographies.

FIG. 2A: Control cells showing hexagonal regular distribution representing tight junctions (X25).

FIG. 2B: 20 min after VEGF incubation. Disruption of tight junctions (arrows) and punctate irregular occludin pattern at the membrane (arrowheads) are observed (X25).

FIG. 2C: 24 hours after VEGF incubation. Increased rupture of tight junctions (arrows), occludin distribution from the membrane to the cytoplama and nulcei (stars) c:Inset:X50. Punctate irregular occludin pattern at the membrane (arrowheads).

FIG. 2D: 65 hours after VEGF incubation. Recovery of occludin membrane localization with remaining cytoplasmic occludin (stars) (X25).

FIG. 3: Effect of hypoxic conditions (10 nM CoCl2) on VEGF, VEGFR-1 and VEGFR-2 expression and on TER of ARPE-19 monolayers.

FIG. 3A: VEGF RT-PCR products from ARPE-19 cells, 16 hours after exposure to various concentrations of CoCl2. Results are expressed as means±SD of VEGF/b-actin ratio (n=3 separate experiments).

FIG. 3B: Time course of VEGFR-1 expression after exposure to 10 nM CoCl2. Results are expressed as means±SD of VEGFR-1/b-actin ratio (n=3 separate experiments).

FIG. 3C: Time course of VEGFR-2 expression after exposure to 10 nM CoCl2. Results are expressed as means±SD of VEGFR-2/b-actin ratio (n=3 separate experiments).

FIG. 3D: Time course of TER monitoring after exposure of ARPE-19 monolayers to 10 nM CoCl2. Results are expressed as percentage of mean net TER in comparison with mean TER before exposure to CoCl2. Means±SD result from three separate experiments.

FIG. 4: Effect of PlGF-1, agonist of VEGFR-1 or J-IgG, an agonist of VEGFR-2 on ARPE-19 monolayers permeability

FIG. 4A: Time course of TER decrease after incubation with 1 nM VEGF 165 or 2 nM PlGF-1. Net TER results from the difference between 10 measures of TER cell monolayer and TER filter alone. Results are expressed as percentage of net TER in comparison with net TER before exposure to VEGF or PlGF-1. Mean±SD result from three separate experiments

FIG. 4B: Time course of inulin flux across ARPE-19 monolayers after incubation with 1nM VEGF 165 or 2 nM PlGF-1. ³H-inulin flux was measured by counting radioactivity (indicated by cpm) in 10 μl during a period of 10 to 180 minutes. Data are representative of 3 experiments.

FIG. 4C: Time course of inulin flux across ARPE-19 monolayers after incubation with 4 nM J-IgG, a specific agonist of VEGFR-2. ³H-inulin flux was measured by counting radioactivity (indicated by cpm) in 10 μl during a period of 10 to 180 minutes. Data are representative of 3 experiments.

FIG. 5: Effect of antisense oligonucleotides directed at either VEGFR-1 or VEGFR-2 on ARPE-19 monolayers VEGF(1 nM)-induced permeability

FIG. 5A: ARPE-19 cells monolayers were incubated with lnMVEGF after 24 hours of pre treatment with antisense oligonucleotides directed at VEGFR-1. Net TER results from the difference between 10 measures of TER cell monolayer and TER filter alone. Results are expressed as percentage of mean net TER in comparison with mean TER before exposure to VEGF. Mean±SD result from three separate experiments

FIG. 5B: ARPE-19 cells monolayers were incubated with 1M VEGF after 24 hours of pretreatment with antisense oligonucleotides directed at VEGFR-2. Net TER results from the difference between 10 measures of TER cell monolayer and TER filter alone. Results are expressed as percentage of mean net TER in comparison with mean TER before exposure to VEGF. Mean±SD result from three separate experiments.

FIG. 5C: Down-regulation of VEGFR-1 expression by specific antisense oligonucleotides, three hours after 10 nM CoCl2 exposure. No effect of the scrambled oligonucleotides was observed on VEGFR-1 expression.

FIG. 5D: Down-regulation of VEGFR-2 by specific antisense oligonucleotides, there hours after 10 nM CoCl2 exposure. No effect of the scrambled oligonucleotides was observed on VEGFR-2 expression.

FIG. 6: Zonula Occludens immunostaining on whole flat-mounted RPE.

FIG. 6A: 5 hours after intravitreal BSS (balance Saline Solution) injection.

FIGS. 6B and 6C: 5 hours after PLGF-1 (100 nM) intravitreal injection. Cell junctions are not more observed and a diffuse cytoplasmic distribution of zonula occludens protein is recognized. Spaces can be distinguished in between RPE cells (Arrows).

### EXAMPLE 1

### 1.1 Materials and Methods

ARPE-19 cell Culture

ARPE-19 (ATCC CRL-2302), a spontaneous immortalized human RPE cell line (Dunn et al., Exp Eye Res. 1996 February; 62(2):155-169) was cultured in a mixture 1:1 of Dulbecco's modified Eagle's medium (DMEM) and Nutrient Mixture F12, with Hepes buffer (Gibco, Grand Island, N.Y., U.S.A.) supplemented with 10% fetal bovine serum (FBS). ARPE-19 cells were seeded at a density of 1.5×10⁵ cells/cm² onto either 25 cm² flasks (Becton Dickinson, USA) or on transwell polyester filters (0.4 μm pore size, 6.5 mm in diameter) (Transwell; Costar, Cambridge, Mass.). Seeding at this cell density on transwells allowed for the formation of monolayers. The cultures were kept at 37° C. in 5% CO₂ humidified atmosphere.

### Treatment of ARPE-19 Cultures

VEGF165 (J. Plouet J Biol. Chem. 1997 May 16; 272(20):13390-6) was added at concentrations ranging from 0.15 to 1 nM/culture. To determine which receptor does mediate VEGF-induced permeability, confluent ARPE-19 monolayers grown on transwells were incubated either with 2 nM PlGF-1 (agonist for VEGFR-1) (Relia Tech GmbH, Braunschweig, Germany) or with 4 nM J-IgG, an agonist of VEGFR-2 (Ortega et al., 1997, Am J Pathol 151:1215-24). For transepithelial electrical resistance (TER) assessments and for inulin flux measurements, modulators were added to serum-free medium. To mimic hypoxic conditions, ARPE-19 monolayers were incubated with CoCl2 as previously described (Piret et al., Ann N Y Acad. Sci. 2002 November; 973:443-7).

### Transepithelial Electrical Resistance Measurements

Experiments were performed on confluent ARPE19 cells monolayers, grown on transwell filters. Changes in transepithelial resistance (TER) across the monolayer were monitored every week until the 4^{th} week of culture using the resistance system (Millicell; Millipore, Bedford, Mass.) connected to dual Ag—AgCl electrodes. Net TER values were calculated by subtracting the mean resistance determined for ten plastic filters in absence of cells from the values recorded for each cell monolayer grown on similar filters. Resistance was expressed in Ohms/cm². After TER stabilization (3 to 4 weeks cultures), various concentrations of VEGF 165 or PlGF-1 were added. Changes in TER were monitored after 1, 10 and 30 minutes, and 1, 6, 24, 36, 48 and 65 hours of incubation with VEGF, PlGF-1 or medium. Δ(TER) corresponds to the difference between TER values of ARPE 19 monolayers after and before stimulation (control cells). Values are illustrated as the mean of three or more independent cultures.

### Permeability Assay by [³H]Inulin Flux

The paracellular permeability of cultured RPE cells was measured by the passive permeation of [³H]inulin across confluent cells grown on filters. The apical and basal sides of the cell layers were bathed in 300 μl and 600 μl serum-free culture media containing the different stimulants and supplemented with 1 mM unlabeled inulin (Sigma France). These incubation volumes were chosen to avoid interference from hydrostatic pressure. [³H]inulin (2 ΞCi; specific activity, 1.95 mCi/millimole) was added to the medium bathing the apical side of the cells. Aliquots of 10 μl were collected from the lower chambers at different time points during 120 minutes. The radioactivity in the collected aliquots was counted in a liquid scintillation analyzer (Packard, Meriden, Conn., USA). All measurements were performed in triplicates and illustrated as the mean counts per minute of test culture.

### Immunohistochemistry on ARPE-19 Monolayers

Confluent RPE cells grown on polyester filters were fixed with 4% parafolmaldehyde in PBS for 20 minutes at room temperature, rinsed and incubated in PBS containing 15% skimmed milk for 15 minutes to block nonspecific binding sites. Cells were incubated for 60′ minutes in a humidified atmosphere at room temperature with a rabbit polyclonal Occludin antibody (Zymed Laboratories, San Francisco, Calif.) in PBS containing 1% skimmed milk. The cells were then rinsed in PBS containing 1% BSA and further incubated for 60 minutes with anti-rat Texas red (Jackson Immuno Research, Baltimore, Pa.). Filters were sealed between glass slide and coverslips and examined under a fluorescence microscope (Leica, Switzerland).

### Expression of VEGF, VEGFR-1 and VEGFR-2 Using Semi-Quantitative RT-PCR

For RT-PCR analysis in normal and hypoxic conditions, cells were grown on 25 cm² flasks. Total mRNA was extracted from each cell culture according to the manufacture's instruction (Qiagen, Valencia, Calif.). The extracted RNA was quantified, and 2 μg of each sample were used to make cDNA using SuperScript 11 (Invitrogen, Life Technologies). RT-PCR quantification was carried out in a linear range. The PCR conditions consisted of denaturation at 95° C. for 30 seconds, annealing at 55° C. (β-actin), 57° C. (VEGFR-1, 2 and VEGF) for 30 seconds and extension at 72° C. for 60 seconds. The reaction was initiated by adding two units of Taq DNA polymerase (Invitrogen), followed by 35 cycles for VEGF, VEGFR-1, 2 and 25 cycles for β-actin. The primers used in this experiment were SEQ ID No4: 5′-CCTCCGAAACCATGAACTTT-3′ (forward), SEQ ID No5: 5′-AGAGATCTGGTTCCCGAAAC-3′ (reverse) for VEGF (637 bp) (Nakayama et al., 2002, Cancer Lett 176:215-223), SEQ ID No6: 5′-CAAGTGGCCAGAGGCATGGAGTT-3′ (forward), SEQ ID No7: 5′-GATGTAGTCTTTACCATCCTGTTG-3′ (reverse) for VEGFR-1 (498 bp) (Masood et al., 1997, Proc Natl Acad Sci USA 94:979-984.), SEQ ID No8: 5′-GAGGGCCTCTCATGGTGATTGT-3′ (forward), SEQ ID No9: 5′-TGCCAGCAGTCCAGCATGGTCTG-3′ (reverse) for VEGFR-2 (706 bp) (Masood et al., 1997, Proc Natl Acad Sci USA 94:979-984.), SEQ No10: 5′-AGGAGAAGCTGTGCTACGTC-3′ (forward) and SEQ No11: 5′-AGGGGCCGGACTCGTCATAC-3′ (reverse) for (β-actin (465 bp).

### Down-Regulation of VEGFR-1 and VEGFR-2 Using Antisense Oligonucleotides

A preliminary experiment was performed to confirm the specificity and efficacy of antisense oligonucleotides (ODN) directed at either VEGFR-1 or VEGFR-2. For this experiment, and in order to have enough material for RNA extraction, cells were seeded on 25 cm² flasks. After 7 days of culture, cells were exposed to CoCl2 (hypoxic conditions) with or without pre treatment with specific antisense ODN. The following phosphorothioate ODN were used: VEGFR-1 antisense: SEQ ID No1: 5′-GTAGCTGACCATGGTGAGCG-3′, VEGFR-1 scrambled: SEQ ID No12: 5′-ATCAGCGTGTGAGCACGGTG-3′, VEGFR-2 antisense: SEQ ID No13: 5′-GCATCTCCTTTTCTGAC-3′ and VEGFR-2 scrambled: SEQ ID No14: 5′-TTAATCGTTCTCTGCCC-3′.

Oligonucleotides (8.0 μg) and 20 μl of Lipofectamine 2000 reagent (Invitrogen) were diluted separately each in 500 μl of OptiMEM I Reduced Serum Medium and incubated at room temperature for 5 min. Oligonucleotides and Lipofectamine 2000 were then mixed and incubated together for another 20 min. This mixture was added to each flask and another 2 ml Opti MEM was added to each flask to prevent from dryness, and then cells were incubated at 37° C. for 5 hours. The transfection mixture was then gently removed and replaced with DMEM containing 10% FBS. 24 hours later 10 μM CoCl2 was added for three hours. The cells were then collected for RNA extraction and VEGF receptors expression analysis using RT-PCR as previously described.

Antisense oligonucleotides were then used to pre treat confluent ARPE-19 monolayers before TER measurements under VEGF stimulation. Oligonucleotides (0.8 μg) and 2.0 μl of Lipofectamine 2000 reagent (Invitrogen) were diluted separately, each in 50 μl of OptiMEM I Reduced Serum at room temperature for 5 min. Oligonucleotides and Lipofectamine 2000 were then mixed and incubated together for another 20 min. This mixture was then added to the cells in 200 μl Opti MEM and incubated at 37° C. for 5 hours. After 5 hours, The transfection mixture was gently removed and replaced by DMEM 2% FBS for 24 h before TER monitoring under 1 nM VEGF stimulation as previously described.

### Effect of Intravitreous Injection of PlGF-1 on RPE Cells Junctions Using ZO-1 Immunohistochemistry on Flat-Mounted RPE/Choroid/Sclera.

To confirm the effect of PlGF-1 on RPE cells junctions in vivo, female Lewis rats, 6-7 weeks old and weighing 150-200 g (IFFA CREDO, Lyon, France) were used. Experiments were conducted in accordance with the ARVO Statement for the Use of Animals in Ophthalmic and Vision Research. Rats were held in the vivarium for 1 week before inclusion in the study.

For the experimentation, rats were anesthetized by intraperitoneal pentobarbital injection (40 mg/kg). They received a single intravitreous injection of 10 ng/ml (n=4) PlGF-1 diluted in 5 μl of PBS or 5 μl of PBS alone (n=2). Other control rats (n=2) did not receive any injection. Five hours after PlGF-1 injection, rats were sacrificed by an overdose of pentobarbital, and rat RPE tight junctions were labeled by immuno-localization of the tight junction-associated protein zonula occludens-1 (ZO-1). Eyes were enucleated, fixed in 2% paraformaldehyde for 1 hour. Biomicroscopic dissection of the sclera, choroid and RPE complex was achieved after removal of the cornea and extirpation of the lens, vitreous body and retina. The sclera, choroid and RPE complex was washed three times for 5 minutes in 1% bovine serum albumin/phosphate-buffered saline (BSA/PBS) then washed three times for 10 minutes in 1% BSA/PBS and 0.2% saponin and subsequently incubated overnight at 4° C. with rat anti-ZO-1 antibody (1:200, Chemicon, Temecula, Calif.) diluted in 1% BSA/PBS and 0.2% saponin. ZO-1 was visualized by incubation with Alexa-conjugated goat anti-rat IgG (1:100, Molecular Probes, Eugene, Oreg., USA). The complex was finally mounted with great care to prevent folding using glycerol and observed using appropriate filters using a confocal microscope (Zeiss L S M 510, Switzerland).

### Statistical Analysis

All experiments were repeated at least three times. The results are expressed as mean±standard deviation. Statistical analysis was performed using the two-tailed Student's t test, P<0.05 was considered significant.

### 1.2 Results

### Effect of VEGF on RPE Tight-Junctions and Permeability

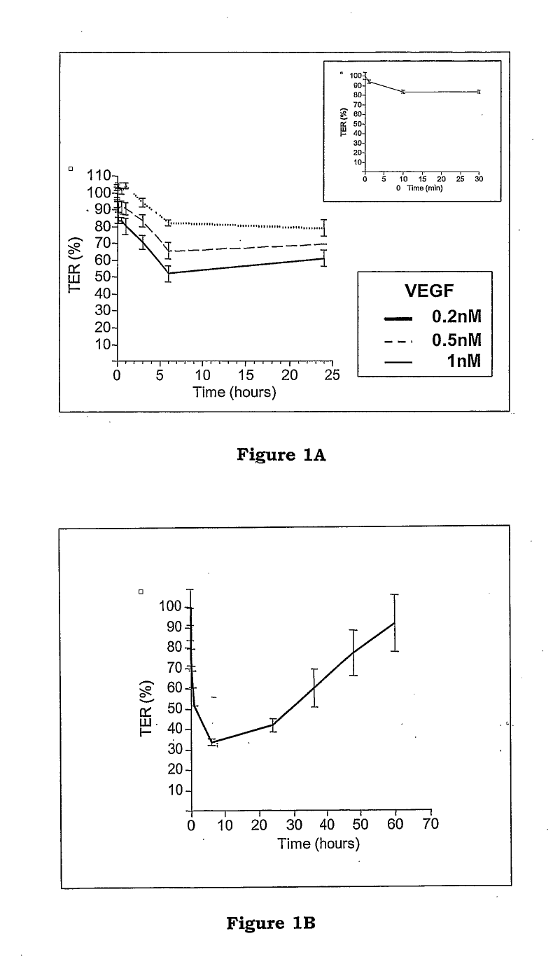

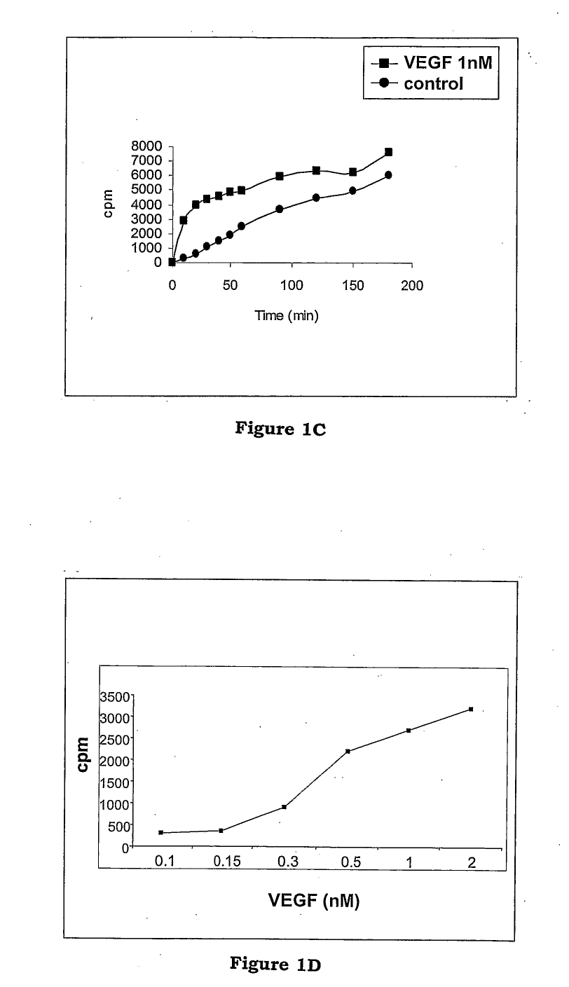

After 4 weeks of culture on transwells, ARPE-19 cells have formed confluent monolayers with a constant mean TER level of 60±8.6 Ohm/cm². Addition of 0.2 nM to 1 nM VEGF induces a dose-response decrease of RPE TER (FIGS. 1A and 1D). The TER decrease is observed as early as 10 min after exposure to 1 nM VEGF (FIG. 1A, inset), accentuating during the first 6 hours, and then slowly returning to normal values in about 65 hours (FIG. 1B). The decrease of TER in cells being suggestive of an alteration of paracellular permeability, flux of inulin, a well-known paracellular tracer was studied. This inert hydrophilic molecule is a small enough extracellular probe to permeate the tight junctions network in RPE cells. Inulin flux through RPE monolayers was recorded as a function of time over 180 min (FIG. 1C). Treatment of RPE cells with VEGF (1 nM) increases the radioactivity measured in the basal side of the well, when compared to non-treated cells (P<0.05). The inulin flux increase across stimulated RPE monolayers was maximal as early as 10 min, lasted for 60 min, and then slowly faded.

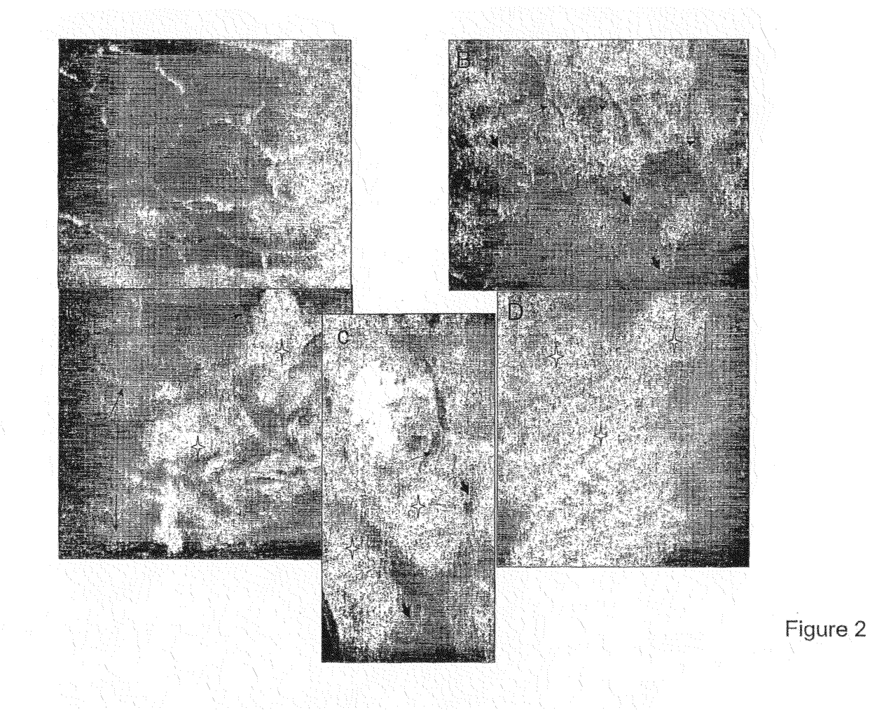

The TER measurements and [³H] inulin fluxes strongly suggest that VEGF may affect the paracellular permeability of RPE tight-junctions. Because changes in tight-junction structures may be counterpart of functional modulations, VEGF-stimulated ARPE-19 monolayers were examined to determine alteration of tight-junction associated proteins. After 4 weeks of culture untreated ARPE-19 monolayers shows continuous occludin labeling around the cell periphery (FIG. 2A). As early as 20 minutes after VEGF addition, and more markedly after 24 hours, discontinuous occludin labeling is observed at the periphery of the cells, spaces between cells can be observed showing unsealed junctions (FIGS. 2B and C, arrows). A change in occludin distribution is observed with labeling in the cytoplasm and the nuclei of cells associated to reduced labeling at the cell membrane (FIGS. 2B and C, stars). Irregular punctate occludin pattern can be observed at high magnification at the membrane of some cells (FIGS. 2B and C, arrowheads). At 65 hours after VEGF addition (1 nM), the initial regular distribution of occludin in the tight junction is reforming with some remaining occludin localization in the cytoplasm (FIG. 2D).

### Effect of Hypoxic Conditions on VEGF and VEGF Receptors Expression, and RPE Permeability

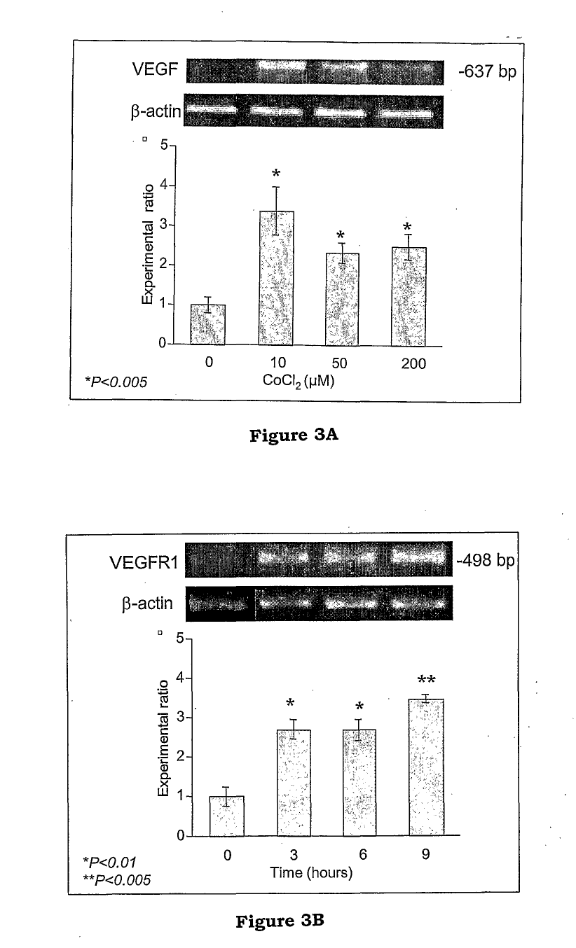

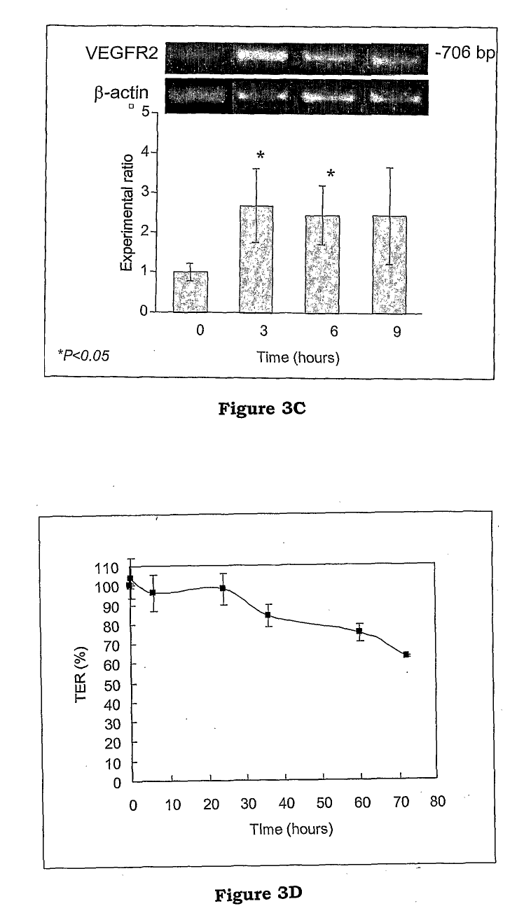

VEGF expression is up-regulated by hypoxia, as confirmed by exposure of ARPE-19 cells to Cocl2. Indeed, VEGF expression is significantly up-regulated sixteen hours after CoCl2 exposure (mimicking hypoxic conditions) (10, 50 and 200 μM) (p<0.005 for all doses) (FIG. 3A). The inventors therefore have examined the influence of hypoxic conditions of VEGF receptors expression and RPE monolayers permeability. VEGFR-1 and VEGFR-2 are expressed in RPE cells in culture under basal conditions (FIGS. 3A and 3B) and in cells with tight junctions (not shown). Under hypoxic conditions VEGFR-1 expression is already up-regulated at 3 and 6 hours (p<0.01) with increased expression 9 hours after exposure (p<0.005). VEGFR-2 expression is also up-regulated under these conditions at 3 and 6 hours (p<0.05), but decreasing thereafter (FIG. 3C). Since hypoxia induces an up-regulation of VEGF receptors, the inventors have followed the changes in ARP-19 TER as a function of time after exposure to hypoxic conditions (10 μM CoCl2). Exposure of cells to 10 μM CoCl2 induces a delayed but prolonged decrease of TER, beginning at 24 hours after exposure and lasting for at least 3 days (FIG. 3D) suggesting that endogenously produced VEGF may modulate RPE cells permeability in vitro.

### Effect of Specific Agonists of VEGF Receptors or Antisense Oligonucleotides Directed at VEGF Receptors on VEGF-Induced RPE Paracellular Permeability.

In order to determine which VEGF receptor, VEGFR-1 or VEGFR-2 is mediating the VEGF-induced paracellular permeability, the inventors have used two different strategies. They examined the effect on TER RPE monolayers of PlGF-1, agonist of VEGFR-1 and of J-IgGI, an agonist of VEGFR-2 (Ortega et al., 1997, Am J Pathol 151:1215-24). In a separate experiment, they examined the potentially inhibitory effect on VEGF-induced TER decrease, of RPE pre treatment with antisense oligonucleotides directing at either VEGFR-1 or VEGFR-2.

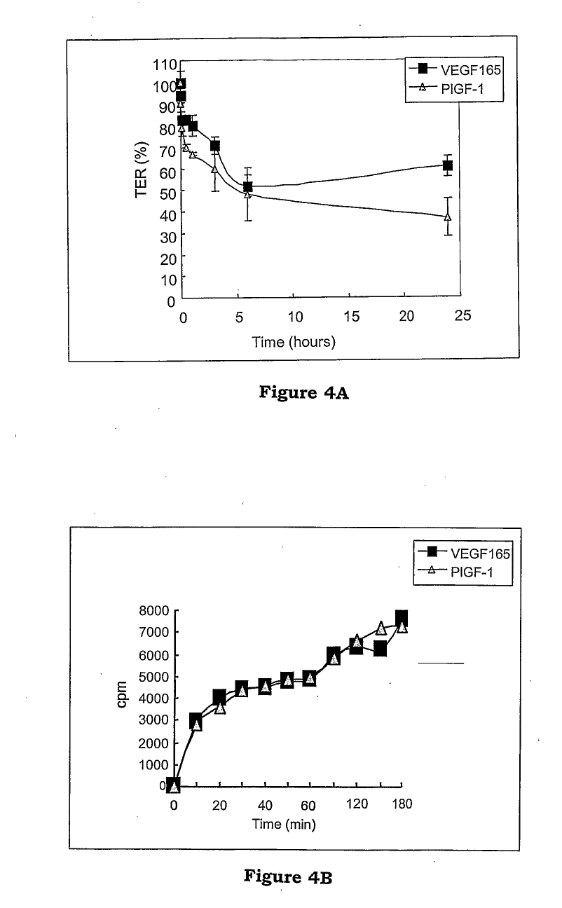

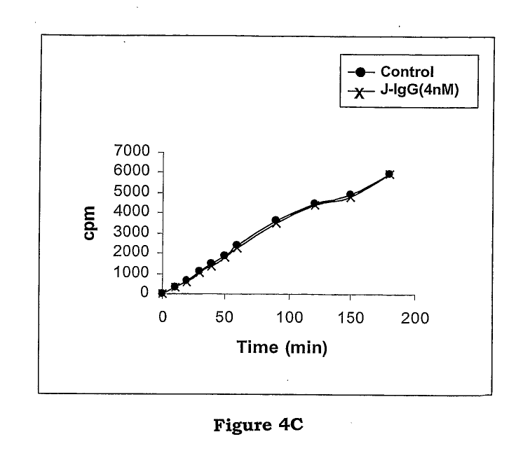

PlGF-1 (2 nM) induces a decrease in RPE TER monolayer, similar in kinetics and intensity to the one induced by VEGF 1 nM (FIG. 4A and FIG. 4B). On the other hand, J-IgG I, an agonist of VEGFR-2 does not induce any decrease of TER (not shown), and does not increase the flux of [3H] inulin across RPE monolayers demonstrating that activation of VEGFR-2 does not seem to influence paracellular permeability (FIG. 4C).

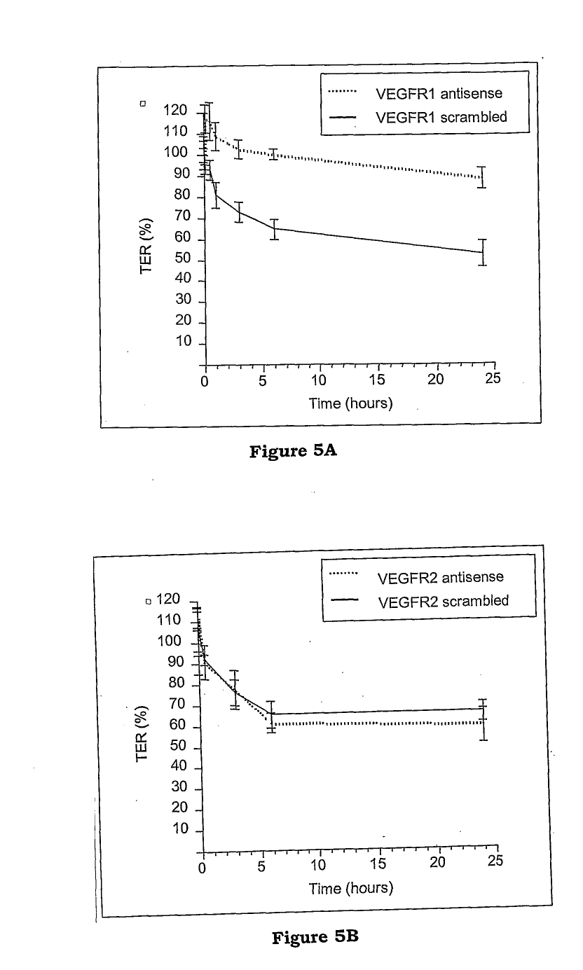

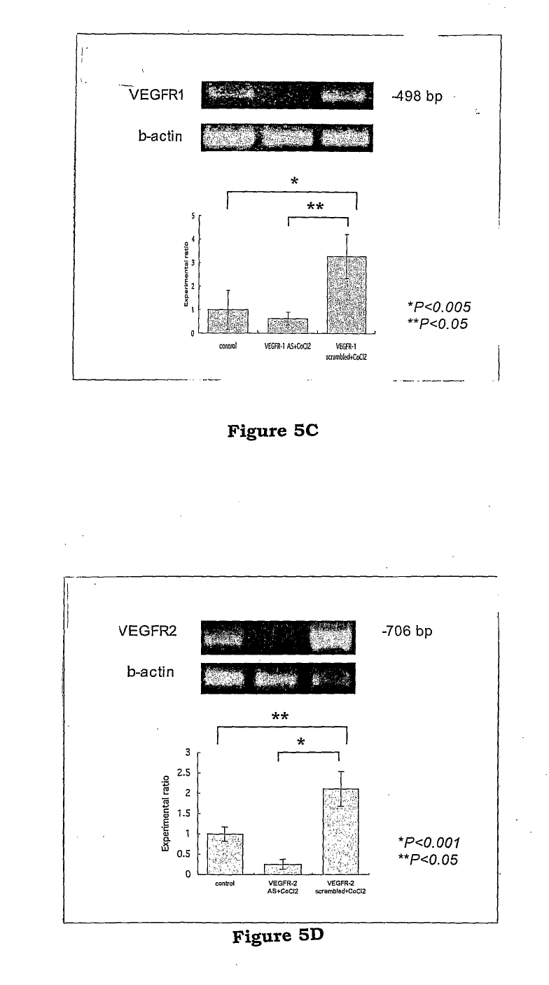

On the other hand, twenty-four hours of RPE cells pretreatment with an antisense oligonucleotide directed at VEGFR-1 abolishes the effect of VEGF (1 nM) on TER RPE monolayers. No inhibitory effect is observed when the scrambled oligonucleotide is used (FIG. 5A). Pretreatment of RPE cells with an antisense directed at VEGFR-2 does not influence the effect of VEGF on RPE cells TER (FIG. 5B). The efficacy and specificity of these oligonucleotides was confirmed by the specific down regulation of VEGF receptors under hypoxic conditions. The antisense oligonucleotides directed at either VEGFR-1 or VEGFR-2 significantly down-regulated the expression of the receptors, three hours after exposure to 10 μM CoCl2. Scrambled oligonucleotides failed to impede the expression of either of the VEGF receptors (FIGS. 5C and 5D).

### Effect of PlGF-1 on Rat RPE Tight Junctions In Vivo

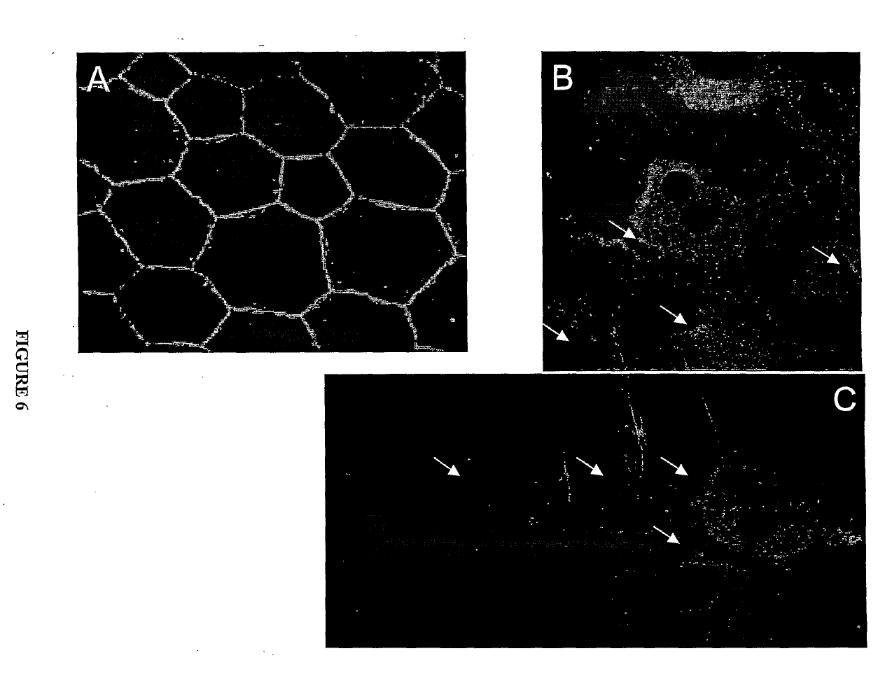

In order to confirm the potential effect of VEGF through VEGFR-1 on RPE cells paracellular permeability in vivo, the inventors have injected PlGF-1 in the vitreous of rats and have looked for any changes in tight-junction associated protein distribution by immunolocalization on RPE flat-mounts 5 hours later (according to the in vitro kinetics). ZO-1 immunohistochemistry on whole flat-mounted RPE/choroids/sclera shows regular localization of all around the cell membranes in control rats RPE (not shown) and in PBS-injected rats (FIG. 6A). A very different pattern of ZO-1 distribution was observed on RPE cells of rats at 5 hours after PlGF-1 (100 ng/ml, 2 nM) intravitreous injection. In the later, spaces between cells and irregular punctate ZO-1pattern is observed associated to a cytoplasmic distribution of the tight-junction associated protein (FIG. 6B).

### 1.3 Discussion

The results show that VEGF modulates tight-junctions of human RPE cells in vitro. The expression of VEGF and its receptors is up-regulated under hypoxic conditions. However, while VEGFR-1 expression is sustained, expression of VEGFR-2 is transitory. These findings suggest that VEGF, produced in retinal diseases in response to hypoxia may influence not only the internal hemato retinal barrier (retinal endothelial cells), but also the external hemato retinal barrier, contributing to the pathogenesis of macular edema and maybe to the growth of neo vessels from the choricapillaries through the RPE.

PlGF-1 which specifically binds to VEGFR-1 (Christinger et al., 2004, J Biol Chem 279:10382-10388), mimics VEGF-induced TER decrease in ARPE-19 in vitro, and induces a structural changes of tight-junctions in the rat eye in vivo. This suggests that the effect of VEGF on RPE permeability could be mediated mostly by Flt-1 and that it does occur in vivo. The inventors have found that J-IgG, a specific VEGFR-2 agonist, had no effect on inulin flux, reinforcing the hypothesis that VEGF-induced paracellular permeability on RPE cells is not mediated by VEGFR-2 but rather by VEGFR-1.

The present result demonstrates that VEGF may regulate the integrity of the external hemato-retinal barrier by a direct effect on tight junctions, and that this regulation is mostly mediated by VEGFR-1. The facts that hypoxic conditions increases permeability of RPE cells in vitro and that PlGF-1 is able to open tight junctions of the rat RPE in vivo are in favor of a real implication of this mechanism in the pathogenesis of retinal diseases associated to VEGF up regulation.

1-18. (canceled)

19. A method for preventing or treating a subject suffering from an ocular disease wherein disorders of the retinal pigment epithelium (RPE) permeation are involved, comprising administering to a subject in need thereof an effective amount of an agonist or an antagonist of vascular endothelial growth factor receptor (VEGFR-1).

20. The method according to claim 19, wherein the ocular disease is selected from the group consisting of macular edema, central serous chorioretinopathy, epitheliopathy and age related macular degeneration (ARMD).

21. The method according to claim 19, wherein the agonist of VEGFR-1 is selected from the group consisting of vascular endothelial growth factor (VEGF) and agonists comprising those originating from Trimeresurus flavoridis venom.

22. The method according to claim 19, wherein the agonist of VEGFR-1 is the placenta growth factor (PlGF).

23. The method according to claim 19, wherein the antagonist of VEGFR-1 is selected from the group consisting of blocking antibodies, MF1 antiflt1mAb, peptides, DHFR-F56/F90, oligoaptamer, ribozymes, siRNA, antisense oligonucleotides, immunomodulator compounds, FTY720, 5-[N-methyl-N-(4-octadecyloxyphenyl)-acetyl]amino-2-methylthiobenzoic acid, and acetyl salicilate.

24. The method according to claim 23, wherein the antagonist is an antisense oligonucleotide of mRNA encoding VEGFR-1, said antisense oligonucleotide having the following sequence:
a) the sequence SEQ ID NO: 1, or
b) a sequence having at least 70% of identity with SEQ ID NO: 1, or
c) a sequence comprising the sequence as defined in a) or b), or
d) a sequence complementary to the sequence as defined in a), b) or c).

25. The method according to claim 19, wherein the subject is a mammal.

26. A pharmaceutical composition comprising an antisense oligonucleotide of mRNA encoding VEGFR-1, said antisense oligonucleotide having the following sequence:
a) the sequence SEQ ID NO: 1, or
b) a sequence having at least 70% of identity with SEQ ID NO: 1, or
c) a sequence comprising the sequence as defined in a) or b), or
d) a sequence complementary to the sequence as defined in a), b) or c), together with a pharmaceutically acceptable carrier.

27. The pharmaceutical composition according to claim 26, for preventing or treating a subject suffering from an ocular disease wherein disorders of RPE permeation are involved.

28. The pharmaceutical composition according to claim 27, wherein the ocular disease is selected from the group consisting of macular edema, central serous chorioretinopathy, epitheliopathy and ARMD.

29. A pharmaceutical composition containing a first compound which is an agonist or an antagonist of VEGFR-1 and a second active compound as a combined preparation for simultaneous, separate or sequential use to treat an ocular disease.

30. The pharmaceutical composition according to claim 29, wherein the ocular disease is selected from the group consisting of macular edema, central serous chorioretinopathy, epitheliopathy, ARMD, infections, intraocular inflammations, retinal dystrophy, diabetic retinopathy, ischemic retinopathy, optic neuritis and ocular diseases associated to neo vascular and/or fibro glial proliferations.

31. The pharmaceutical composition according to claim 29, wherein the ocular disease is an ocular disease wherein disorders of RPE permeation are involved.

32. The pharmaceutical composition according to claim 29, wherein the agonist is selected from the group consisting of VEGF, agonists originating from Trimeresurus flavoridis venom, and specific agonists identified by idiotypic technologies.

33. The pharmaceutical composition according to claim 29, wherein the agonist is PlGF.

34. The pharmaceutical composition according to claim 29, wherein the antagonist is selected from the group consisting of blocking antibodies, peptides, oligoaptamer, ribozymes, siRNA, antisense oligonucleotides, immunomodulator compounds, 5-[N-methyl-N-(4-octadecyloxyphenyl)-acetyl] amino-2-methylthiobenzoic acid and acetyl salicilate.

35. The pharmaceutical composition according to claim 29, wherein the second active compound is selected from the group consisting of anti angiogenic compounds, non steroidal anti inflammatory, cortico corticoids, mineralo corticoids, anti-oxidants, lipids, neurotrophic factors, glial cell line-derived neurotrophic factor (GDNF), brain-derived neurotrophic factor (BDNF), fibroblast growth factor (FGF), COX inhibitors and antibiotics.

36. A method for preventing or treating a subject from having from an ocular disease, wherein a first compound modulates intra-ocular penetration of a second compound which is systemically administered, comprising administering an effective amount of an agonist compound or antagonist compound of VEGFR-1 and said second compound to a subject in need thereof.

37. The method according to claim 36, wherein the ocular disease is selected from the group consisting of macular edema, central serous chorioretinopathy, epitheliopathy, ARMD, infections, intraocular inflammations, retinal dystrophy, diabetic retinopathy, ischemic retinopathy, optic neuritis, and ocular diseases associated with neo vascular and/or fibro glial proliferations.

38. The method according to claim 36, wherein the agonist is selected from the group consisting of VEGF and agonists comprising those originating from Trimeresurus flavoridis venom.

39. The method according to claim 36, wherein the agonist is PlGF.

40. The method according to claim 36, wherein the antagonist is selected from the group consisting of blocking antibodies, MF1 antiflt1mAb, peptides, DHFR-F56/F90, oligoaptamer, ribozymes, siRNA, antisense oligonucleotides, immunomodulator compounds, FTY720, 5-[N-methyl-N-(4-octadecyloxyphenyl)-acetyl]amino-2-methylthiobenzoic acid, and acetyl salicilate.

41. The method according to claim 36, wherein the second compound is selected from the group consisting of anti angiogenic compounds, non steroidal anti inflammatory, cortico corticoids, mineralo corticoids, anti-oxidants, lipids, neurotrophic factors, ciliary neurotrophic factor (CNTF), glial cell line-derived neurotrophic factor (GDNF), brain-derived neurotrophic factor (BDNF), fibroblast growth factor (FGF), COX inhibitors and antibiotics.

42. A method for treating or preventing a subject from having an ocular disease. wherein disorders of the RPE permeation are involved, comprising administering to a patient in need thereof, a pharmaceutical composition comprising an agonist or an antagonist of VEGFR-1 together with a pharmaceutically acceptable carrier.

43. A method for treating or preventing a subject from having an ocular disease comprising administering simultaneously, separately or sequentially to said subject a combined pharmaceutical preparation comprising a first compound which is an agonist or an antagonist of VEGFR-1 and a second active compound together with a pharmaceutically acceptable carrier.
